(19) United States
(12) Patent Application Publication
(10) Pub. No.: US 2004/0244068 A1
(43) Pub. Date: Dec. 2, 2004

(54) Diagnostic marker

(76) Inventors: Paul Richard Heaton, Leicestershire (GB); Brigitte E.H. Smith, Leicestershire (GB); John Merrit Rawlings, Leicester (GB)

Correspondence Address: FULBRIGHT & JAWORSKI, LLP, 1301 MCKINNEY, SUITE 5100, HOUSTON, TX, 77010-3095, US

(21) Appl. No.: 10/484,261

(22) PCT Filed: Jul. 19, 2002
(86) PCT No.: PCT/GB02/03315

(30) Foreign Application Priority Data
Jul. 20, 2001 (GB) 0117790.6

(51) Int. Cl. C12Q 1/68; A01K 67/027

(52) U.S. Cl. 800/14; 435/6

(57) ABSTRACT

The present invention relates to a non-human animal life stage classification system, particularly, but not exclusively using a physiological marker of an individual non-human animal. The life stage classification can be used to more accurately define the life-stage of companion animals.

### CROSS-REFERENCE TO RELATED APPLICATIONS

This application is a national stage application of PCT/GB02/03315 filed July 19, 2002 claiming priority to GB 0117790.6 filed Jul. 20, 2001.

### TECHNICAL FIELD

The present invention relates to a non-human animal life stage classification system, particularly, but not exclusively using a physiological marker of an individual non-human animal. The life stage classification can be used to more accurately define the life-stage of companion animals.

### BACKGROUND OF THE INVENTION

Currently, animals are “aged” using approximate equivalent ages to humans. Studies in humans divide individuals into infant, adult and senior life-stage groups without any clear definition of how these divisions are made. Likewise, canine studies have defined Labrador dogs with a mean age of 2.4 years as being young, those with a mean age of 5.8 as middle aged and those with a mean age of 7.1 as old. Alternatively definitions have been presented for German Shepherd dogs as young, adult and old with no clear guidelines of how these groups were defined.

This “aging” is arbitrary and does not reflect physiological changes which represent changes in the life-stage of the animal. There is a consensus, that the function of the mammalian immune system changes quite dramatically with age. Recent data would suggest that immunosenescence is a more complex process than previously envisaged. Thymic involution, commonly thought to be the major causative factor in age related immune deficiency, may not play as central a role as initially perceived. Some studies have shown that certain aspects of immune function, in particular elements of the more evolutionary distant innate response, seem to be preserved or possibly upregulated well into old age. This suggests that immunosenescence may be more accurately viewed as an immune remodeling process rather than a simple deterioration.

Accordingly, there is a need to define a life-stage classification, for non-human animals. Such a classification system can be used to determine the effects, of various factors, on aging.

### BRIEF SUMMARY OF THE INVENTION

In any life-stage classification, a marker of change is required. The present inventors have determined that a particular marker is a good measure to define life-stages. This marker provides a better life-stage classification than previous markers such as weight or other physical measurements.

Accordingly, the present invention provides, according to a first aspect, the use of a physiological marker to determine the life-stage of a non-human animal.

The present inventors have determined that significant age-related changes in several physiological parameters, reflect an altered functional status of the canine and feline animal. Using these changes, life-stage classifications were identified. This is the first report which has based life-stage classifications on a physiological marker and opens up the potential for designing specific life-stage diets based on changing physiological status with age. The present invention provides a platform to monitor development, maintenance and modulation of the canine or feline physiological system with age.

The present invention provides novel strategies (in particular utilizing nutritional intervention) to enhance the ability of the physiological system to cope with stressors (a disorder which has a component of oxidative stress).

These disorders include cancer, aging, heart disease, atherosclerosis, arthritis, cataracts, inflammatory bowel disease, renal disease, renal failure, neurodegenerative disease or compromised immunity, for example, an animal suffering from an infection.

Using one or more indicators of changing physiological function to define life-stage groups (puppy/kitten, adult and senior) provides the ability to balance nutritional requirements for a particular life-stage to help maintain optimal physiological function, or help boost a sub-optimal physiological system and potentially reduce the incidence of stressors which are associated with aging.

The present inventors have identified that changes in the immune status occur at distinct times in the life of canine and feline animals.

In the present invention, the physiological marker may be one or more indicators of immune status. Such indicators include one or more cell-mediated immune marker, one or more antibody mediated immune marker or one or more innate immune marker.

Particular cell mediated immune markers, according to the present invention, include T-cell markers such as populations of CD3 cells, CD4 cells, CD8 cells, CD21 cells, CD11b cells, granulocytes and lymphocytes. The ratio of CD4:CD8 cells is also a useful marker.

In the first aspect of the invention, the life-stage may be defined by any means. It can be by time (i.e. age) or by division into puppy/kitten, adult and senior areas (or others).

The present invention applies primarily to non-human animals, in particular to companion animals. Typical companion animals include cats and dogs, such as the domestic dog (Canis domesticus) and the domestic cat (Felis domesticus). All breeds are encompassed in the present invention. Any life-stage classification may cover all species or may be species (i.e. breed) specific. For example, the life stage classification may be for Labrador or German Shepherd dogs, specifically. It may also relate for example to large breed dogs versus small breed dogs.

The second aspect of the present invention relates to a method for determining the life-stage of a non-human animal, the method comprising determining the population of one of the following T-cells in an individual animal, and with reference to the indicated figure, determining the life stage of the animal:

For a canine animal;

CD3: FIG. 1a or FIG. 2a

CD4: FIG. 1b or FIG. 2b

CD8: FIG. 1c or FIG. 2c

CD4:CD8 ratio: FIG. 1d or FIG. 2d

CD21: FIG. 1e or FIG. 2e.

For a feline animal;

CD4: FIG. 3

CD8: FIG. 4 or FIG. 9

CD4:CD8 ratio: FIG. 5

Granulocytes: FIG. 6

Lymphocytes: FIG. 7

CD11b cells: FIG. 8.

Significant age-related changes were seen to occur in the T and B parameters measured. CD3 and CD8 were the most significantly affected parameters. Highly significant changes were determined between animals less than 8-12 months of age, between 1-8 years of age and greater than 8 years of age. Accordingly, the following life-stage classification is presented according to the present invention:

| Life-stage | Life-stage in years | |
|---|---|---|
| Puppy/kitten | <8-12 | months of age |
| Adult dog/cat | 1-8 | years of age |
| Senior dog/cat | >8 | years of age |

These life stages have been established on the basis of the following immune markers:

| Life-stage | Life-stage in years |
|---|---|
| Puppy/kitten | CD4:CD8 ratio |
| Adult dog/cat | CD4:CD8 ratio, or CD3, CD8, or CD21 |
| Senior dog/cat | CD4:CD8 ratio, CD3 or CD8 or CD21 |

All preferred features of the first aspect also apply to the second.

A third aspect of the present invention provides a non-human animal life-stage classification system comprising a figure, chart or a table which relates the level of a physiological marker of an individual animal to a particular life stage for the animal.

Examples of figures can be found in any of the FIGS. 1a, 1b, 1c, 1d 1e, 2a,2b, 2c, 2d, 2e, 3, 4, 5, 6, 7, 8 and 9.

As for all aspects of the invention, the physiological marker may be any which has been shown to be associated with the aging process. As well as markers which reflect immune status, others which are suitable include markers of the cell-cycle, DNA repair, antioxidant markers etc.

All preferred features of aspects one and two, also apply to the third.

A fourth aspect of the present invention provides the use of a life-stage classification, according to the third aspect of the invention, to determine the effect of one or more factors on the physiological marker. The physiological marker may be any, particularly those described for the first to third aspects of the invention.

The factor to be determined is not limiting. Suitable factors include: diet, medicine, environmental change (seasons or geographical change), stress etc.

A particular feature of the fourth aspect of the invention is to determine the effect of diet. The term “diet” here includes nutrition and covers all foodstuffs which the animal ingests. It includes the traditional basic foodstuffs, as well as supplements, drinks, snacks, treats etc. The foodstuff includes wet diets, dry diets and semi-moist diets.

In accordance with the present invention, a life-stage classification can be used to determine the effect of diet. In this manner, diet can be identified which has a beneficial effect on the animal. Accordingly diets can be identified which restore declining immune status.

Dietary intervention can thus be provided for a beneficial effect. The diet may need to be life-stage specific. Or, it may be species specific (breed species) such as Labrador dogs or large breed versus small breed specific. The diet may be both life-stage and species (or other) specific. Inter-breed differences in life-span may have a major impact on immune status with age, leading to/supporting different diets for different breeds. Nutritional supplementation (e.g. vitamin C) or combinations of supplements or other diet can be used to maintain optimal immune performance, or rejuvenate age-related reduced/suppressed immune function in cat and dog populations. From a clinical perspective, baseline data from a healthy population can be used for comparative purposes with populations that are clinically immunosuppressed to assess the effectiveness of clinical diets.

All preferred features of the first to third aspects also apply to the fourth.

The fifth aspect of the invention relates to a companion animal life-stage classification as set out in any one of FIGS. 1a, 1b, 1c, 1d, 1e, 2a, 2b, 2c, 2d, 2e, 3, 4, 5, 6, 7, 8 or 9.

A sixth aspect of the invention provides a method of feeding a non-human animal comprising:

(a) measuring a physiological marker of an animal;

(b) determining a life-stage classification for the animal in dependence upon said measurement; and

(c) supplying the animal with a diet which varies according to the life-stage classification.

All preferred features of the first to fourth aspects also apply to the fifth.

A seventh aspect of the invention provides a method of feeding a non-human animal, substantially as hereinbefore described, with reference to one or more of FIGS. 1a, 1b, 1c, 1d, 1e, 2a, 2b, 2c, 2d, 2e, 3, 4, 5, 6, 7, 8 or 9.

An eighth aspect of the invention provides an animal foodstuff optimized for a specific life-stage classification of a non-human animal, the said classification being determined according to average measurements of a physiological marker made on a sample population of animals of a species or breed by which the feed is to be consumed.

In the eighth aspect of the invention the average measurements of a physiological marker referred to include typical or representative measurements of said physiological marker.

The animal foodstuff according to the eighth aspect of the invention may be optimized for nutrition of an animal of said classification.

The animal foodstuff of the aspect of the invention may be optimized to enhance the physiology of an animal of said classification. Enhancing the physiology may well enable the animal to cope with stresses typical of said classification.

Enhancing the physiology of an animal may include enhancing representative physiological markers of said animal.

An eighth aspect of the invention may comprise an animal foodstuff optimized to enhance the physiology of an animal of said classification to cope with the disorders typical of said classification.

The animal foodstuff according to the eighth aspect of the invention may comprise a dog foodstuff or a cat foodstuff.

The animal foodstuff according to the eighth aspect of the invention may be optimized, or further optimized for animals of a particular size-range, a particular weight or a particular breed.

An animal foodstuff of the eighth aspect of the invention may comprise the foodstuff being packed within a container which carries a visible indicator of said classification for which it is optimized.

According to the present invention, the non-human foodstuffs encompass any project that a non-human animal consumes in its diet. Specifically companion animal or pet animal foodstuffs are included. Thus, the invention covers the standard food products as well as food snacks (for example snack bars, cereal bars, snacks, biscuits and sweet products). Such food snacks may be pet food snacks. The foodstuff may be a cooked product. It may incorporate meat or animal-derived material (such as beef, chicken, turkey, lamb, fish, blood plasma, marrowbone, etc or one or more thereof). The foodstuff may alternatively be meat-free (preferably including a meat substitute such as soya, maize gluten or a soya product) in order to provide a protein source. The foodstuff may contain additional protein sources such as soya protein concentrate, milk, protein, gluten etc. The foodstuff may also contain a starch source such as one or more grains (e.g. wheat, corn, rice, oats, barley, etc) or may be starch-free. The foodstuffs may incorporate or may be a gelatinized starch matrix. The foodstuff may incorporate one or more types of fibber such as sugarbeet pulp, chicory, coconut endosperm fibber, wheat fibber etc.

The foodstuff may be a dry, semi-moist or a moist (wet) product. Wet food includes food that is usually sold in tins and has a moisture content of 70% to 90%. Dry food includes food having a similar composition but with 5% to 15% moisture, often presented as small biscuit-like kibbles. Semi-moist food includes food having a moisture content of from above 15% up to 70%. The amount of moisture in any product may influence the type of packaging that can be used or is required.

The foodstuff is preferably packaged. In this way, the consumer is able to identify, from the packaging, the ingredients in the foodstuff and confirm that it is suitable for the particular non-human animal in question. The packaging may be metal (usually in the form of a tin or flexifoil), plastic (usually in the form of a pouch or bottle), paper or card. The amount of moisture in any product may influence the type of packaging, which can be used or is required.

### BRIEF DESCRIPTION OF THE DRAWINGS

The invention is described with reference to the Figures, in which:

FIG. 1a shows a scatter plot of relative percentages of CD3 cells in relation to age (years). A linear regression analysis is provided.

FIG. 1b shows an equivalent plot, where the cells are CD4 cells.

FIG. 1c shows an equivalent plot, where the cells are CD8 cells.

FIG. 1d shows an equivalent plot, where the cells are a ratio of CD4:CD8.

FIG. 1e shows an equivalent plot, where the cells are CD21 cells.

FIG. 2a is a discriminant analysis plot of CD3 cell populations from dogs, divided into those of up to 8 years and those over 8 years.

FIG. 2b shows an equivalent plot, where the cells are CD4 cells.

FIG. 2c shows an equivalent plot, where the cells are CD8 cells.

FIG. 2d shows an equivalent plot, where the cells are CD4:CD8 ratio.

FIG. 2e shows an equivalent plot, where the cells are CD21 cells.

FIG. 3 shows a scatter plot of relative percentages of CD4 cells in relation to age (years). A linear regression analysis is provided.

FIG. 4 shows an equivalent plot, where the cells are CD8 cells.

FIG. 5 shows an equivalent plot where the cells are a ratio of CD4:CD8 ratio.

FIG. 6 shows an equivalent plot where the cells are granulocytes.

FIG. 7 shows an equivalent plot where the cells are lymphocytes.

FIG. 8 shows an equivalent plot where the cells are CD11b cells.

FIG. 9 is a discriminant analysis of the CD4:CD8 ratio data divided into those up to 8 months and those of over 8 months of age.

### DETAILED DESCRIPTION OF THE INVENTION

The present invention will now be described with reference to the following examples.

### Example 1

Materials and Methods

Animals

Whole blood specimens were taken from 122 Labrador dogs (71 females and 51 males) ranging from 0.6 years to 14.2 years in order to establish immunological baseline data on age-related changes in peripheral blood leukocyte subsets.

Sample Collection

All blood samples were collected into a 1 ml potassium EDTA tube for fluorescence activated cell sorting (FACS) analysis.

Leukocyte Profile Determination

The immune response is composed of a variety of cell-types (leukocytes) with differing finctions. Identification of these different leukocyte populations provides a foundation for understanding the basis of the immune response. With the development of monoclonal antibodies (Mab) with reactivity against leukocyte populations, markers for total T-cells (CD3), T-cell subsets (CD4 and CD8), B-cells (CD21) and monocytes (CD14) represent some of the measurable populations.

FACS analysis has been widely used for characterizing and quantifying viable sub-populations of white blood cells. FACS analysis involves three steps; Firstly, cells are prepared and incubated with the relevant Mab against a particular surface marker and labeled with a fluorescent reagent (such as fluorescein isothiocyanate (FITC)). Secondly, stained cells are identified and separated by the FACS and the data collected. Thirdly, the collected data are analyzed to obtain quantitative information on the relevant cell populations.

Canine Leukocyte Populations

Relative expression levels of the following immunological cell surface markers and leukocyte populations were determined by using lysed whole blood staining and triple-color FACS analysis:

| | |
|---|---|
| CD3 | Total T-cell marker |
| CD4 | T-helper cell marker |
| CD8 | T-cytotoxic marker |
| CD4:CD8 ratio | Relative levels of T-helper to T-cytotoxic |
| CD21 | B-cell marker |
| CD14 | Monocyte marker |
| Lymphocytes | Subpopulation of leukocytes |
| Granulocytes | Subpopulation of leukocytes |
| Monocytes | Subpopulation of leukocytes |
| Lymphocyte viability | Measure of live to dead cells |
| C11b | Cell surface marker |

cells

Statistical Analysis

Values were expressed as percentages of cellular populations for each individual animal. The results were statistically evaluated by linear regression to assess trends over the whole age ranges. Discriminant analysis was used to identify the subgroup of cell surface markers that were most successful at discriminating between the derived life-stage groupings. For differences between life-stages an independent sample t-test was performed on each of the individual cellular populations to identify significant differences between life-stages.

### Results

Canine Leukocyte Populations

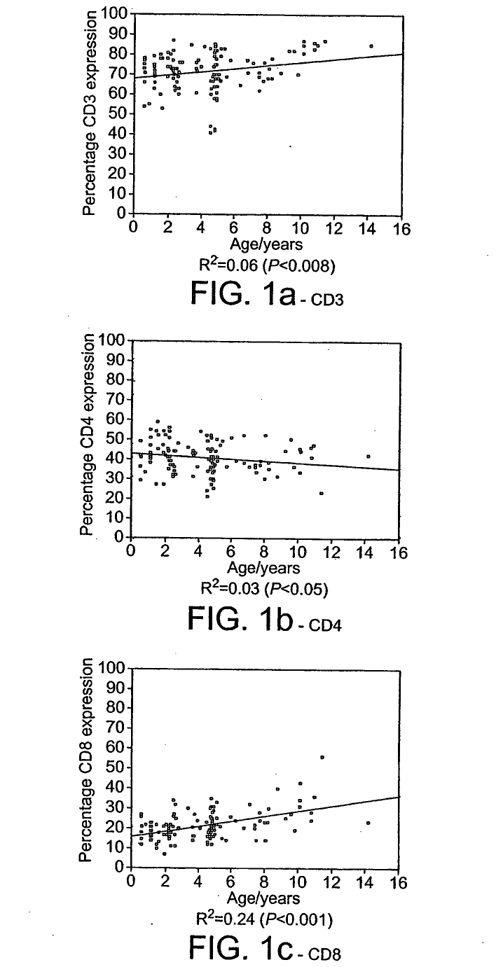

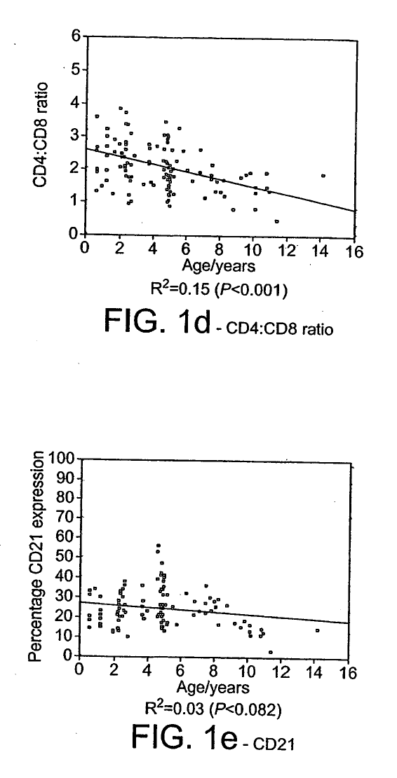

The following scatter plots show the relative percentages of different leukocyte populations in relation to age (years). Linear regression analysis was used to identify significant trends in the data obtained. Analysis identified a significant increase in CD3 (R²=0.06, p<0.008; FIG. 1a), significant decrease in CD4 (R²=0.03, p<0.05; FIG. 1b), a significant increase in CD8 (R²=0.24, p<0.001; FIG. 1c), with a corresponding decrease in the CD4:CD8 ratio (R²=0.15, p<0.001; FIG. 1d) with increasing age.

Age groups for discriminant analysis is at 8 years of age

Canine Life-stage Groups

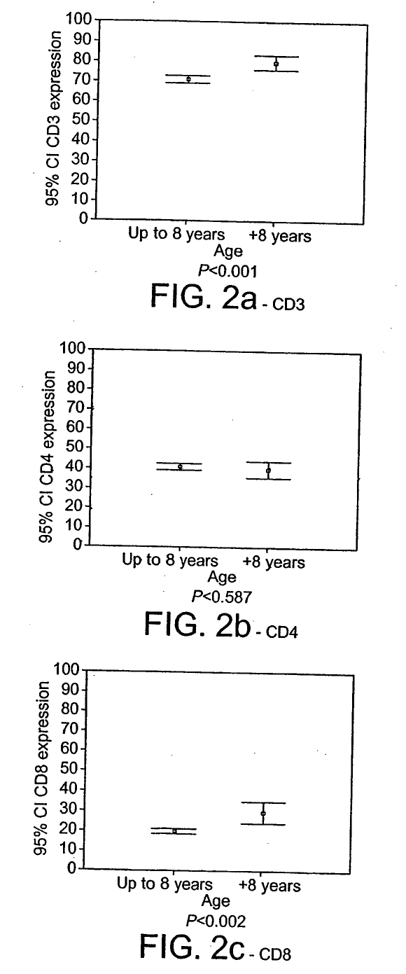

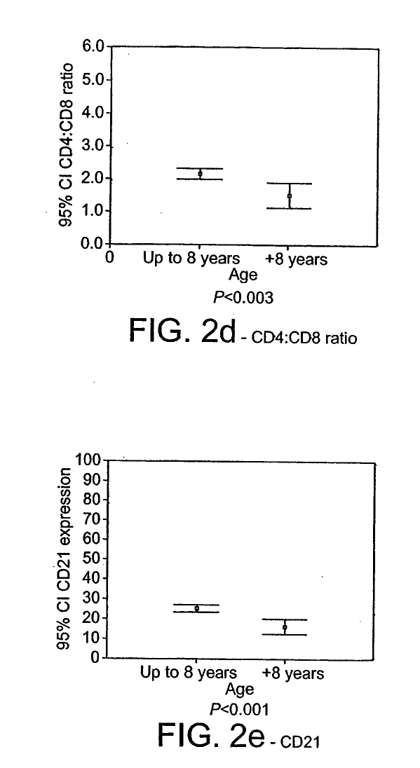

These results illustrate a significant correlation between aging and change in relative percentages of several leukocyte groups in the Labrador dog population. Using discriminant analysis to identify leukocyte populations that were most successful at discriminating between the derived life-stage groupings, CD3 (FIG. 2a) and CD8 (FIG. 2c) defined two statistically distinct groups, adult dogs (0.6 to 8 years) and senior dogs (8+ years), with an overall correct classification of 83% (cross-validated) for both markers.

### Discussion

Canine Leukocyte Populations

Our investigations have demonstrated significant age-related increases in the relative percentages of CD3 and CD8 T-cells, and decreases in CD4 T-cells and the CD4:CD8 ratio in peripheral blood from the dog population.

Significant changes observed in the present study were with T-cell markers (CD3, CD4, CD8, and CD4:CD8 ratio), all of which are associated with the cellular component of the acquired immune system (response of antigen-specific T-cells to antigen, including development of immunological memory). Although some of the R² values are particularly low in some cases (FIG. 1a, 1b), this is not unexpected as these data represent the natural variability seen in a population when taking independent samples of healthy dogs over an age range of 0.6 to 14.2 years of age.

The CD3 T-cell marker, which defines total T-cells (encompassing both the CD4 and CD8 T-cell subgroups) demonstrated a significant increase in relative percentage with age (FIG. 1a). This may partly be a result from a relative increase in CD8 T-cells that co-express the CD3 marker being greater than a relative decrease in CD4 T-cells that express the same marker. Studies in humans have also demonstrated an increase in levels of CD8 T-cells with age suggesting that these cell types correspond to immature T-cells that are unable to attain their full mature functional status due to the age-associated processes highlighted above. Also, when combined with interpretation of data from other canine and human studies which have examined absolute and relative values of total T-cell subsets, it was shown that an increase in percentage of total T-cells is apparent because the absolute numbers of the T-cell subsets are declining at a lower rate.

While the CD3 marker is commonly shared by the CD4 and CD8 T-cells, their functions are very distinct from each other. The function of CD8 T-cells (cytotoxic T-cells) is to identify and destroy host cells that contain intracellular pathogens (e.g. viruses). CD4 T-cells (helper T-cells) are specialized to activate other cells to destroy extracellular pathogens (e.g. bacteria and parasites) and fall into two functional classes: T-helper 1 (TH1) cells which activate monocyte/macrophages to kill bacteria they harbor, and TH2 cells, which activate B-cells to produce antibody.

The significant increase in relative percentage of canine CD8 T-cells (FIG. 1c) observed could form part of what is termed the “memory” T-cell population. As mentioned above, production of memory T-cells indicates that the body has reacted to each antigenic stimulus and mounted a response to suppress or eliminate that particular infection. Over time these same physiological responses lead to a progressive accumulation of (expanded) clones of memory T-cells that allow the individual to respond more quickly to an infection if that particular antigen is encountered again. This supports the finding that levels of CD8 T-cells increase with age.

In conclusion, studies on measuring functional cellular aging combined with analyses of cells within the aging individual provides new insights on how modulation of the immune system develops with age. Evidence from the dog population has demonstrated age-related changes in relative percentages of leukocyte populations in peripheral blood. These studies suggest that continuous restructuring occur in distinct leukocyte populations, potentially altering the functional status of the aging canine immune system. Information from such studies provides the basic platform from which to monitor the plasticity of the immune system, overall maintenance of basal immunity over time, regulation of compensatory functions and modulation of long-term memory. This information will undoubtedly help develop novel strategies (in particular utilizing nutritional intervention) to enhance the ability of the immune system to cope with such stressors (as herein before described) during the life of an animal, in particular during the latter stages of an animal's life.

From the information present, it is clear that significant changes do occur in the canine immune status, showing that there is modulation of immune status with age. Using discriminant analysis on the scatter plot information for each of the leukocyte groups, both the CD3 and CD8 T-cell markers defined two statistically distinct life-stage groups, adult dogs below 8 years of age and senior dogs above 8 years of age (FIGS. 2a, c). Although the CD4:CD8 ratio (FIG. 2d) and CD21 (FIG. 2e) showed significant differences, the classification was not as high as for CD3 and CD8.

The possible reason for the lower classification level of the CD4:CD8 ratio is because of the negligible difference in the percentage of CD4 cells between the two life-stage groups (FIG. 2b). This may be attributed to the reduced number of dogs at the higher end of the age scale (>8 years), but also the percentage of CD4 cells in the senior group may have reached a basal level just below the level of the adult group, thus eliminating seeing any significant reductions over 8 years of age.

Although the decline in percentage of CD21 cells was not significant (FIG. 1e), the significant difference between the adult and senior life-stage groups (FIG. 2e), albeit at a lower classification level, may indicate that a greater reduction in percentage of CD21 cells occurs around the 8 years of age level, which in turn would be masked when put in the overall context of the age scatter plot profile.

Such indicators of changing immune status between different life-stages can be used to determine whether an animal is undergoing immune dysfunction, with the consequences being reduced immune surveillance for infections and potential cancer-inducing factors with increasing age. Information of this kind is useful to define nutritional requirements for a particular life-stage to maintain optimal immune function, or to help boost a sub-optimal immune system and potentially reduce the incidence of degenerative disorders.

### Example 2

### Materials and Methods

Whole blood samples were taken from 288 Domestic Shorthaired cats, 121 males and 167 females ranging from 0.2 to 15.9 years o1d. All cats were housed in conditions resembling those of pet cats and were maintained on commercially available complete diets.

Samples were analyzed using lysed whole blood staining and two-color flow cytometric analysis. Commercially available monoclonal antibodies were used to identify cell surface markers for CD5, CD4, CD8, B-cells (CD21-like), CD14 and CD1lb. Relative levels of lymphocytes, monocytes and granulocytes were also calculated.

Values were expressed as percentages of cellular populations for each individual animal. Age-related trends were assessed by linear regression analysis. Discriminant analysis and independent sample t-test were used to identify cellular populations for defining life-stage groupings.

Results

Linear regression analysis identified:

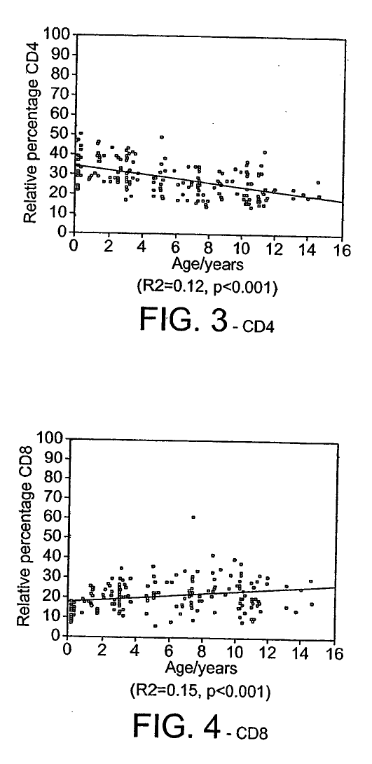

A significant decrease in CD4 (R²=0.12, p<0.001; FIG. 3).

A significant increase in CD8 (R²=0.15, p<0.001; FIG. 4).

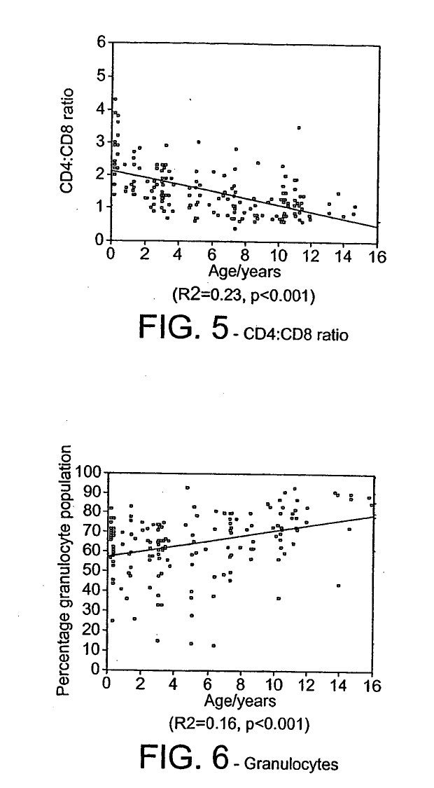

A significant decrease in CD4:CD8 ratio (R²=0.23, p<0.001; FIG. 5).

A significant decrease in relative levels of granulocytes (R²=0.16, p<0.001; FIG. 6).

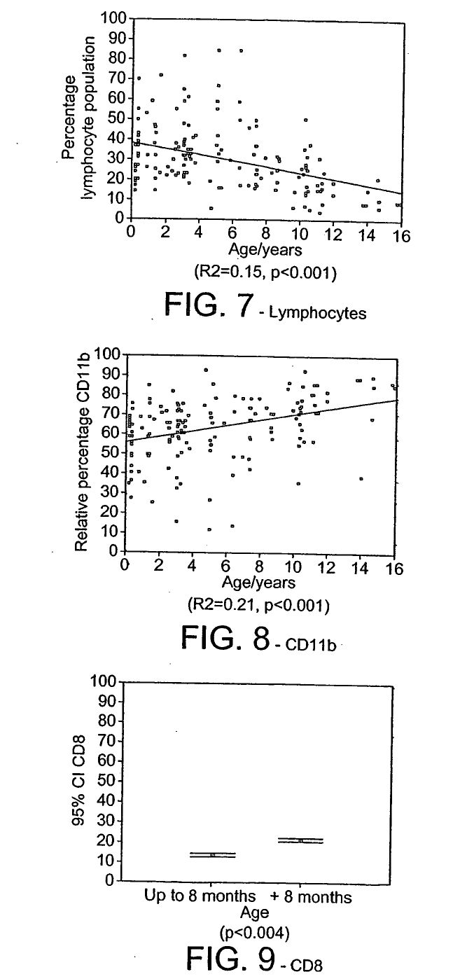

A significant decrease in relative levels of lymphocytes (R²=0.15, p<0.001; FIG. 7).

A significant increase in CD11b (R²=0.21, p<0.001; FIG. 8).

Discriminant analysis of the CD4:CD8 ratio data (FIG. 8) allowed identification of two statistically distinct groups of cats, kittens (0.2 to 0.8 years) and adults (0.8+ years), with an overall correct classification of 77% (cross-validated).

Discussion

Results from this study have demonstrated:

A significant age-related decrease in the relative percentage of CD4

A significant age-related increase in the relative percentage of CD8

A significant age-related decrease in the CD4:CD8 ratio

A significant age-related increase in the relative percentage of CD11b

### CONCLUSIONS

These studies are the first which comprehensively investigate the effects of aging on leukocyte subsets in healthy cats and furthermore to define life-stages based on immunological status. The life-stages identified in this study highlight the fact that immunological status does change according to age in the Domestic Shorthaired cat. Therefore, it is imperative that age be considered in any study where the interpretation of leukocyte subset data is utilized.

1. A method of determining the life-stage of a non-human animal comprising the step of measuring physiological marker.

2. The method of claim 1, wherein the physiological marker is one or more indicators of immune status.

3. The method of claim 2, wherein the immune status is one or more of a cell-mediated immune marker, an antibody-mediated immune marker or an innate immune marker.

4. The method of claim 1, wherein the physiological marker is one or more of the following T-cell populations of an individual animal CD3,CD8,CD4,CD4:CD8 ratio, CD21,granulocytes, lymphocytes or CD11b.

5. The method of claim 1, wherein the life-stage is expressed as age.

6. The method of claim 1, wherein the animal is a companion animal.

7. The method of claim 6, wherein the companion animal is a cat or a dog.

8. A method for determining the life-stage of a non-human animal, the method comprising the step of measuring a population of one or more cell populations selected from the group consisting of CD3, CD4, CD8, CD4:CD8 ratio, CD21, granulocytes lymphocytes and CD11b in an individual animal and, determining the life-stage of said animal based upon the measured cell population.

9. The method of claim 8, wherein the life-stage is expressed as age.

10. A non-human animal life-stage classification system comprising a chart or a table which relates the level of a physiological marker of an individual animal to a particular life stage for the individual animal.

11. The life-stage classification system, of claim 10, wherein the non-human animal is a companion animal.

12. The life-stage classification system, of claim 11, wherein the companion animal is a cat or a dog.

13. The life-stage classification system, of claim 10, wherein the physiological marker is immune status.

14. The life-stage classification system, of claim 10, wherein the immune status is one or more of a cell-mediated immune marker, an antibody-mediated immune marker or an innate immune marker.

15. The life-stage classification system of claims 10, wherein the immune status is one or more of the following cell populations CD3, CD4, CD8, CD4:CD8 ratio, CD21, granulocytes, lymphocytes or CD11b.

16. A method of determining the effect of one or more factors on a physiological marker by using the life stage classification system of claim 10.

17. The method of claim 16, wherein the factor is diet.

18. A companion animal life-stage classification, comprising a table or chart that identifies trends of a physiological marker over age ranges, wherein the physiological marker is selected from the group consisting of CD3, CD4, CD8, CD4:CD8 ratio, CD21, granulocytes, lymphocytes and CD11b.

19. A method of feeding a non-human animal comprising the steps of:
(a) measuring a physiological marker of an animal;
(b) determining a life-stage classification for the animal in dependence upon said measurement; and
(c) supplying the animal with a diet which varies according to the life-stage classification.

20. The method of claim 19, wherein the physiological marker is one or more indicators of immune status.

21. The method of claim 20, wherein the immune status is one or more of a cell-mediated immune marker, an antibody-mediated immune marker or an innate immune marker.

22. The method of claim 19, wherein the physiological marker is one or more of the following cell populations of an individual animal CD3, CD8,CD4. CD4:CD8 ratio, CD21, granulocytes, lymphocytes or CD11b.

23. The method of claim 19, wherein the animal is a companion animal.

24. The method of claim 23, wherein the companion animal is a cat or a dog.

25. (Canceled)

26. An animal foodstuff optimized for a specific life-stage classification of a non-human animal, said classification being determined according to average measurements of a physiological marker made on a sample population of animals of a species or breed by which the feed is to be consumed.

27. The animal foodstuff of claim 26, wherein the foodstuff is optimized for nutrition of an animal of said classification.

28. The animal foodstuff of claim 26, wherein the foodstuff is optimized to enhance the physiology of an animal of said classification.

29. The animal foodstuff of claim 26, wherein the foodstuff is optimized to enhance the physiology of an animal of said classification to cope with disorders typical of said classification.

30. An The animal foodstuff of claim 26 comprising a dog foodstuff.

31. The animal foodstuff of claim 26 comprising a cat foodstuff.

32. The animal foodstuff of claim 26 comprising dry, wet or semi-moist foodstuff.

33. The animal foodstuff of claim 26, wherein the foodstuff is optimized for animals of a particular size-range.

34. The animal foodstuff of claim 26, wherein the foodstuff is optimized for animals of a particular weight.

35. The animal foodstuff of claim 26, wherein the foodstuff is optimized for animals of a particular breed. 36. The animal foodstuff of claim 26, wherein the foodstuff is packed within a container which carries a visible indicator of said classification for which it is optimized.
